(19) United States
(12) Patent Application Publication
(10) Pub. No.: US 2024/0331359 A1
(43) Pub. Date: Oct. 3, 2024

(54) LEARNING DATA SET GENERATION METHOD, MACHINE LEARNING MODEL, IMAGE PROCESSING DEVICE, LEARNING DATA SET GENERATION DEVICE, MACHINE LEARNING DEVICE, IMAGE DIAGNOSIS SYSTEM, AND PROGRAM

(71) Applicant: Konica Minolta, Inc., Tokyo (JP)

(72) Inventors: Shikou KANEKO, Saitama (JP); Hiroaki MATSUMOTO, Kanagawa (JP); Yoshihiro TAKEDA, Tokyo (JP)

(21) Appl. No.: 18/609,382

(22) Filed: Mar. 19, 2024

(30) Foreign Application Priority Data
Mar. 27, 2023 (JP) 2023-050355

(51) Int. Cl. G06V 10/774; G06T 7/00; G06V 10/82; G16H 50/20

(52) U.S. Cl. CPC G06V 10/774; G06T 7/0012; G06V 10/82; G16H 50/20; G06T 2207/10088; G06T 2207/10132; G06T 2207/20081; G06T 2207/20084; G06V 2201/03

(57) ABSTRACT

Provided is a learning data set generation device of the present disclosure, which causes a computer to execute: acquiring second medical imaging data generated by predetermined image conversion processing on first medical imaging data; and generating a pair of the second medical imaging data and a first ground truth label, which is a ground truth label for the first medical imaging data, as a learning data set.

### CROSS REFERENCE TO RELATED APPLICATIONS

The entire disclosure of Japanese Patent Application No. 2023-050355 filed on Mar. 27, 2023, is incorporated herein by reference in its entirety.

### BACKGROUND

### Technological Field

The present disclosure relates to a learning data set generation method, a machine learning model, an image processing device, a learning data set generation device, a machine learning device, an image diagnosis system, and a program.

### Description of the Related Art

In recent years, there have been attempts in the medical field to use a machine learning model, on which machine learning has been performed, for support of image diagnosis and the like. Machine learning causes a machine to learn patterns and correlations of data by using a large amount of data, thereby performing identification, recognition, detection, prediction, and the like.

Japanese Patent Publication Laid-Open No. 2022-39989 discloses an image processing device which generates a second radiation image in which noise is reduced as compared with a first radiation image by inputting the first radiation image acquired by an acquisition section into a learned model obtained by learning using learning data including a radiation image obtained by adding noise in which a high-frequency component is attenuated.

### SUMMARY

In order to create a highly accurate machine learning model (discriminator) by machine learning, the amount of learning data used for learning and the quality of a ground truth label corresponding to the learning data are important.

Depending on the applications of the machine learning model, it may be difficult to prepare a large amount of learning data. Further, even when a large amount of learning data can be prepared, a lot of time and labor are required to assign a high-quality ground truth label to each of the large amount of learning data. Accordingly, there is a demand for efficiently preparing a learning data set formed of a pair of learning data and a ground truth label.

An object of the present disclosure is to provide a learning data set generation method, a machine learning model, an image processing device, a learning data set generation device, a machine learning device, an image diagnosis system, and a program each capable of efficiently preparing learning data sets including a large amount of learning data and ground truth labels.

To achieve at least one of the abovementioned objects, according to an aspect of the present invention, a learning data set generation method according to an aspect of the present disclosure causes a computer to execute: acquiring second medical imaging data generated by image conversion processing on first medical imaging data; and generating a pair of the second medical imaging data and a first ground truth label, which is a ground truth label for the first medical imaging data, as a learning data set.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a configuration of an image diagnosis system according to an embodiment of the present disclosure;

FIG. 2 is a block diagram illustrating an example of a configuration of a learning data set generation device;

FIG. 3 is a flowchart provided for describing an operation example of the learning data set generation device;

FIG. 4 is a diagram illustrating an example of a hardware configuration of the learning data set generation device;

FIG. 5 is a block diagram exemplifying a configuration of a machine learning device; and

FIG. 6 is a block diagram exemplifying a configuration of an image diagnosis device.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, one or more embodiments of the present disclosure will be described in detail with reference to the drawings.

### [Outline]

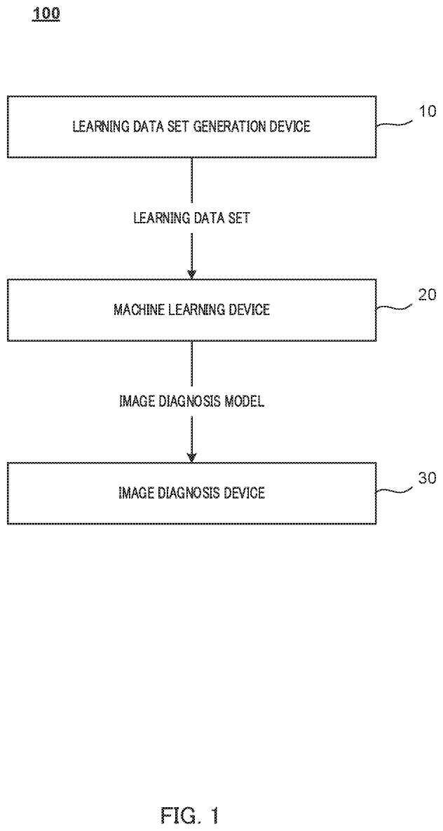

FIG. 1 is a diagram illustrating an example of a configuration of image diagnosis system 100 according to an embodiment of the present disclosure. As illustrated in FIG. 1, image diagnosis system 100 includes learning data set generation device 10, machine learning device 20, and image diagnosis device 30.

Learning data set generation device 10 acquires second medical imaging data generated by predetermined image conversion processing on first medical imaging data, and generates, as a learning data set, a pair of the second medical imaging data and a first ground truth label which is a ground truth label for the first medical imaging data. The learning data set generated by learning data set generation device 10 is inputted into machine learning device 20.

The first medical imaging data is medical imaging data to be inputted into learning data set generation device 10. A first ground truth label is associated with the first medical imaging data in advance.

The predetermined image conversion processing in learning data set generation device 10 is executed by, for example, a predetermined image conversion model on which machine learning has been performed. The image conversion model for executing the predetermined image conversion processing may be included in learning data set generation device 10, or may be included in an image conversion device (not illustrated) different from learning data set generation device 10.

The ground truth label is information that is given to the learning data and indicates a ground truth. The content of the ground truth label varies depending on the content of processing to be executed on data into which a machine learning model that has undergone machine learning by using a learning data set formed of a pair of learning data and a ground truth label has been inputted. In the present embodiment, the ground truth label is, for example, at least one of information indicating a position of a region of interest included in medical imaging data which is learning data (for example, coordinates, a region, a boundary, or the like), information indicating a structure of the region of interest (for example, a bone, a blood vessel, a muscle, a nerve, an affected part, or the like), and information indicating whether the region of interest is normal or abnormal (there is no disease or there is a disease).

Machine learning device 20 outputs a learned image diagnosis model on which machine learning has been performed by using the learning data set inputted from learning data set generation device 10. The learned image diagnosis model outputted by machine learning device 20 is inputted into image diagnosis device 30.

Image diagnosis device 30 outputs, as an image diagnosis result, an inference result obtained by inputting third medical imaging data, which is new medical imaging data, into the learned image diagnosis model. For example, a doctor or the like can perform accurate image diagnosis with reference to the image diagnosis result outputted by image diagnosis device 30.

In the present embodiment, image diagnosis device 30 is, for example, an ultrasound imaging device (ultrasound diagnosis device) that includes an ultrasound probe (probe) and generates ultrasound image data based on a reflected ultrasound wave with respect to an ultrasound wave transmitted to a subject. In a case where image diagnosis device 30 is an ultrasound imaging device, the first to third medical imaging data described above are ultrasound image data.

Generally, in the medical field, completely different images are obtained from different modalities. For example, an MRI image captured by a nuclear magnetic resonance (NMR) imaging device and an ultrasound image captured by an ultrasound device are completely different from each other. For this reason, for example, in a case where machine learning is performed on an image diagnosis model for performing image diagnosis based on ultrasound image data, medical imaging data (for example, MRI image data) other than ultrasound image data, which is generated by a modality other than an ultrasound imaging device, cannot be used as learning data.

Further, even in an ultrasound imaging device, when the type of an ultrasound probe (probe) that transmits and receives ultrasound waves, an imaging mode, or the like is changed, the angle of view, resolution, SN ratio in the depth direction, and the like of an obtained ultrasound image become completely different. For this reason, in a case where machine learning is performed on an image diagnosis model for performing image diagnosis based on ultrasound image data obtained by using an ultrasound probe of one type, ultrasound image data obtained by using an ultrasound probe of another type and/or an imaging mode of another type cannot be used as learning data.

For this reason, for example, in order to prepare a learning data set for an image diagnosis model for performing image diagnosis based on ultrasound image data in a specific imaging mode using a specific ultrasound probe, a work of capturing a large number of ultrasound images in the specific imaging mode using the specific ultrasound probe and then assigning a ground truth label to each image is required. However, it takes a lot of time and labor to capture a large number of ultrasound images in a specific imaging mode and assign a high-quality ground truth label to each image.

Further, invasion such as medical exposure can be a problem depending on the type of medical imaging data used in an image diagnosis model, and therefore, in the case of such medical imaging data, the difficulty of preparing a large amount of medical imaging data of a particular type as learning data increases.

Image diagnosis system 100 according to the embodiment of the present disclosure can obtain a large number of learning data sets, which cause an image diagnosis model for performing image diagnosis by using a certain type of medical imaging data to be efficiently learned, with relatively small labor by generating, by learning data set generation device 10, the learning data sets by image conversion processing of various types of medical imaging data. Then, by using the learning data sets generated by learning data set generation device 10, machine learning device 20 can cause an image diagnosis model, which allows accurate image diagnosis to be performed, to be efficiently learned. Further, since image diagnosis device 30 is capable of outputting an image diagnosis result based on new medical imaging data by using the learned image diagnosis model, image diagnosis device 30 is very useful for medical professionals and the like.

### [Details of Each Configuration]

Hereinafter, each configuration included in image diagnosis system 100 will be described in detail.

### <Learning Data Set Generation Device 10>

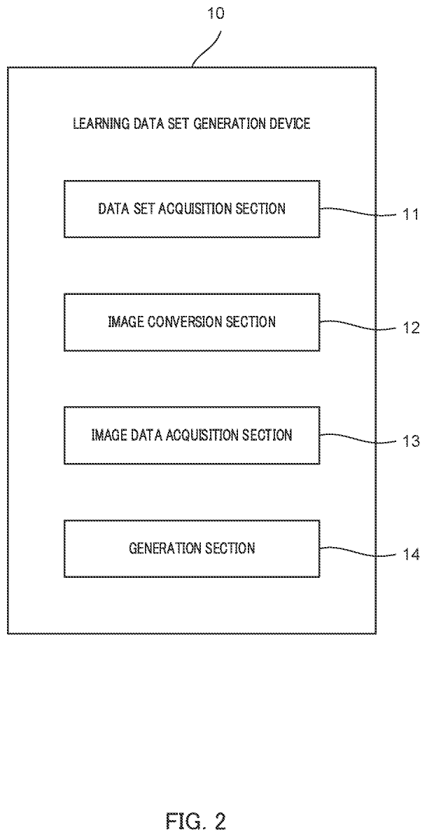

FIG. 2 is a block diagram illustrating an example of a configuration of learning data set generation device 10. In the example illustrated in FIG. 2, learning data set generation device 10 includes data set acquisition section 11, image conversion section 12, image data acquisition section 13, and generation section 14.

Data set acquisition section 11 acquires a data set, which is a pair of first medical imaging data and a first ground truth label, from outside. The outside is, for example, various modalities that generate various medical imaging data, or a database formed of various medical imaging data and ground truth labels.

Image conversion section 12 stores learned image conversion models, and executes predetermined image conversion processing in response to an input of first medical imaging data. The image conversion model is, for example, an image conversion model using a convolutional neural network or an attention mechanism. In the present embodiment, the medical imaging data inputted into image conversion section 12 will be referred to as first medical imaging data, and the medical imaging data converted by image conversion section 12 will be referred to as second medical imaging data. The first medical imaging data may include not one type of imaging data, but each may include various imaging data. The various imaging data include, for example, medical imaging data captured by various modalities, ultrasound image data captured by various ultrasound probes (for example, sector, linear, and convex ultrasound probes) in an ultrasound imaging device, and ultrasound image data captured in various imaging modes.

Note that, in the example illustrated in FIG. 2, a case where learning data set generation device 10 includes image conversion section 12 including an image conversion model is described, but as described above, the present disclosure is not limited thereto. For example, learning data set generation device 10 may acquire second medical imaging data outputted by an external image conversion device based on an input of first medical imaging data.

The image conversion processing executed by image conversion section 12 is, for example, image conversion processing for converting the style of an image, which is called style conversion. The style conversion is a method of extracting style information of a certain image, and transferring and synthesizing only a style and a texture while substantially leaving a prototype of another image. As an image conversion model for performing style conversion, for example, an existing model such as cycleGAN or a diffusion model may be used.

Image conversion section 12 performs style conversion in response to an input of various types of first medical imaging data, and outputs second medical imaging data that appears to be ultrasound image data of a specific type. In this case, the image conversion model included in image conversion section 12 is, for example, a machine learning model on which machine learning has been performed so as to convert various types of first medical imaging data into data such as ultrasound image data for which a linear probe is used. Image conversion section 12 can output various types of second medical imaging data by exchanging the machine learning model. For example, by exchanging the image conversion model included in image conversion section 12, image conversion section 12 may output second medical imaging data that appears to be an MRI image in response to inputs of various first medical imaging data. In addition, image conversion section 12 may include a plurality of types of image conversion models in advance, and may appropriately replace the image conversion model to be used, as necessary (for example, in response to an instruction by the user).

Specific examples of the image conversion processing performed on first imaging data by the image conversion model included in image conversion section 12 will be described.

As a first example, image conversion section 12 converts first medical imaging data generated by using various types of ultrasound probes into second medical imaging data generated by using a specific ultrasound probe in an ultrasound imaging device. Pieces of medical imaging data generated using different types of ultrasound probes have different center frequencies and have different image qualities, that is, spatial resolutions and SN ratios. Image conversion section 12 converts the first medical imaging data generated by one of the three types of ultrasound probes, for example, the sector, linear, and convex ultrasound probes, into the second medical imaging data that appears to be medical imaging data generated by a specific one of the ultrasound probes, for example, the linear ultrasound probe. For example, according to the first example, in a case where it is desired to prepare ultrasound image data obtained by using an ultrasound probe of a type that is not very popular, or the like, ultrasound image data obtained by using an ultrasound probe of a type that is not very popular can be easily prepared by converting ultrasound image data obtained using an ultrasound probe of a type that is relatively popular.

As a second example, image conversion section 12 converts first medical imaging data generated using various types of modalities into second medical imaging data generated using a specific modality. Examples of the various types of modalities include an ultrasound imaging device, a nuclear magnetic resonance imaging device, a computed tomography (CT) device, and an X-ray imaging device. As described above, for example, ultrasound image data generated by using an ultrasound imaging device and MRI image data generated by using a nuclear magnetic resonance imaging device are completely different images. Image conversion section 12 converts, for example, MRI image data generated by using a nuclear magnetic resonance imaging device or CT image data generated by using a CT device into second imaging data that appears to be ultrasound image data.

An MRI image generally has a better SN ratio for a deep part of the image than an ultrasound image. Accordingly, by using MRI image data as the first medical imaging data, it may be possible to acquire a first ground truth label with higher accuracy than in a case where the first medical imaging data is ultrasound image data. After image conversion section 12 converts the first medical imaging data, which is MRI image data, into the second medical imaging data that appears to be ultrasound image data, generation section 14, which will be described later, generates a learning data set by pairing the second medical imaging data and the first ground truth label, thereby improving the quality of the ground truth label in the generated learning data set. Thus, the generalization performance of the image diagnosis model improves, and the diagnostic accuracy improves. Further, by converting the first medical image, which is ultrasound image data, into the second medical image that appears to be an X-ray image, it is possible to obtain a large number of learning data sets without the needs to consider medical exposure or the like for the subject.

As a third example, image conversion section 12 converts first medical imaging data generated by using modalities of the same type (for example, an ultrasound imaging device) manufactured by various manufacturers into second medical imaging data generated by using a modality manufactured by a specific manufacturer. Modalities manufactured by different manufacturers may generate medical imaging data with completely different image qualities even when the modalities are of the same type or even when the modalities capture images of the same subject. Image conversion section 12 converts first medical imaging data generated using modalities manufactured by, for example, Company A, Company B, Company C, and Company D into second medical imaging data that appears to have been generated using a modality manufactured by one specific company, for example, Company C. Thus, the generalization performance of the image diagnosis model improves, and the diagnostic accuracy improves. That is, by including the second medical imaging data in the learning data set, it is possible to train the image diagnosis model so as to be able to cope with more image qualities and more anatomical patterns of subjects.

As a third example, image conversion section 12 converts first medical imaging data generated under various image processing conditions, that is, first medical imaging data having gains, spatial frequencies, and the like different from each other into second medical imaging data that appears to be generated under a specific image processing condition. Thus, the generalization performance of the image diagnosis model improves, and the diagnostic accuracy improves. That is, by including the second medical imaging data in the learning data set, it is possible to train the image diagnosis model so as to be able to cope with more image processing conditions (gain, spatial frequency, and the like) and more anatomical patterns of subjects.

As a fourth example, image conversion section 12 converts first medical imaging data generated by various kinds of signal processing, that is, first medical imaging data generated with different focuses in an ultrasound imaging device into second medical imaging data that appears to be generated with a specific focus. Thus, the generalization performance of the image diagnosis model improves, and the diagnostic accuracy improves. That is, by including the second medical imaging data in the learning data set, it is possible to train the image diagnosis model so as to be able to cope with differences in focus.

As a fifth example, image conversion section 12 converts a plurality of first medical imaging data, in which the BMIs (Body Mass Index) of the subjects of the first medical imaging data are different from each other, into second medical imaging data in which images of a subject having a specific BMI appear to be captured. As a result, for example, first medical imaging data in which a thin subject, a subject having a standard body type, a fat subject, and the like are mixed can be converted into second medical imaging data in which, for example, images of a fat subject appears to be captured. Thus, the generalization performance of the image diagnosis model improves, and the diagnostic accuracy improves. That is, by including the second medical imaging data in the learning data set, it is possible to train the image diagnosis model so as to be able to cope with differences in image quality due to the body shapes of subjects.

As a sixth example, image conversion section 12 converts a plurality of first medical imaging data generated at different transmission voltages into second medical imaging data that appears to be generated at a specific transmission voltage. For example, in an ultrasound imaging device, the image quality of medical imaging data generated by a device with a relatively low transmission voltage (for example, a portable device) may be lower than that of medical imaging data generated by a device with a relatively high transmission voltage (for example, a stationary device). Thus, the generalization performance of the image diagnosis model improves, and the diagnostic accuracy improves. That is, by including the second medical imaging data in the learning data set, it is possible to train the image diagnosis model so as to be able to cope with differences in image quality due to the transmission voltage.

As a seventh example, image conversion section 12 deletes a specific one of various objects reflected in the imaging range of first medical imaging data and converts the first medical imaging data into second medical imaging data. Examples of the various objects reflected in the imaging range of first medical imaging data include an artifact, an artificial structure such as a puncture needle, an anesthetic solution, a contrast medium, and an affected area.

For example, by performing conversion to delete an artificial structure or the like existing outside a region of interest indicated by a first ground truth label associated with first medical imaging data, second medical imaging data without the artificial structure can be generated. Thus, when generation section 14 to be described later generates a learning data set, it is not necessary to newly assign a ground truth label to second medical imaging data obtained by deleting an artificial structure from first medical imaging data. Accordingly, it is possible to omit the labor for providing a ground truth label. Further, even when various objects are reflected other than the region of interest in first medical imaging data, a ground truth label is assigned without being affected by the reflected objects, and thus a high-quality learning data set is generated. Then, by performing training by using the learning data set generated in this way, it is possible to obtain an image diagnosis model that can cope with, with high accuracy, even in a case where various objects (such as an artifact, an artificial structure such as a puncture needle, an anesthetic solution, a contrast medium, or an affected area) reflected in the imaging range of first medical imaging data are not reflected.

Note that, although the case where an artificial structure or the like existing outside the region of interest is deleted has been described in the above-described example, for example, image conversion section 12 may delete a specific structure reflected inside the region of interest.

As an eighth example, image conversion section 12 adds specific data, which is not reflected in the imaging range of first medical imaging data, to the first medical imaging data and converts the first medical imaging data into second medical imaging data. For example, by performing image conversion for newly adding an image of an affected area into the region of interest of the first medical imaging data, it is possible to newly generate second medical imaging data in which the specific affected area is reflected. In addition, it is possible to prepare a learning data set for performing learning for recognizing the same region of interest regardless of the presence or absence of an affected area in the region of interest. Thus, the quality of the learning data set generated by generation section 14 improves, which is more suitable. Note that, specific objects that are not reflected in the imaging range are not limited to an affected area, but may be an artificial structure such as a puncture needle, an artifact (for example, an artifact caused by the presence of a puncture needle), an anesthetic solution, or a contrast medium. By including the second medical imaging data, in which the first medical imaging data is converted such that those described above are included, in the learning data set, it is possible to train the image diagnosis model so as to be able to cope with, with high accuracy, even when an artifact, an anesthetic solution, a contrast medium, or an affected area is reflected in an image.

In the present embodiment, it is assumed that image conversion section 12 performs style conversion without changing a contour position of inputted first medical imaging data. Thus, in response to inputs of various types of first medical imaging data, image conversion section 12 can obtain second medical imaging data converted into one type of medical imaging data in which only the style and texture of a part of the image or the entire image are different, for example, without changing the position of the boundary between tissues such as bones, muscles, and blood vessels.

Image data acquisition section 13 acquires second medical imaging data outputted by image conversion section 12 and inputs the second medical imaging data into generation section 14. Note that, in an example in which learning data set generation device 10 does not include image conversion section 12, image data acquisition section 13 acquires the second medical imaging data from an external image conversion device.

Generation section 14 generates a learning data set for learning an image diagnosis model by pairing second medical imaging data with a first ground truth label. That is, the learning data set generated by generation section 14 includes the second medical imaging data obtained by subjecting the first medical imaging data to the style conversion, and the first ground truth label associated with the first medical imaging data. Generation section 14 outputs the generated learning data set to machine learning device 20.

As described above, a contour position of an image is not changed by the style conversion in image conversion section 12. For this reason, the first ground truth label indicating information on a region of interest or the like in the first medical imaging data is directly applicable to the second medical imaging data obtained by subjecting the first medical imaging data to the style conversion.

As described above, when a pair of first medical imaging data and a first ground truth label is inputted, learning data set generation device 10 performs image conversion processing including the style conversion on the first medical imaging data to generate second medical imaging data of a specific type, and then outputs the pair of the second medical imaging data and the first ground truth label as a learning data set. Here, since the type of the first medical imaging data inputted does not matter, it is easy to prepare a large amount of first medical imaging data. Thus, even when the second medical imaging data after the image conversion processing is medical imaging data of a specific type for which it is difficult to collect a large number of learning data sets, a large amount of second medical imaging data can be obtained by preparing a large amount of various first medical imaging data regardless of the type.

For this reason, a large amount of second medical imaging data of a specific type can be generated.

In addition, since the first ground truth label corresponding to the first medical imaging data before the conversion can be applied as it is as the ground truth label corresponding to the second medical imaging data converted in the image conversion processing, it is not necessary to prepare a ground truth label again even in a case where a large amount of second medical imaging data is generated. Thus, a large number of learning data sets can be obtained without requiring labor.

Further, learning data set generation device 10 does not include the first medical imaging data in the inputted data set (a pair of first medical imaging data and a first ground truth label) in the learning data set to be outputted. Thus, it is possible to generate a learning data set including second medical imaging data of which the type is completely different from that of first medical imaging data (for example, modalities are different or the types of the ultrasound probes are different). Thus, the learning data set outputted by learning data set generation device 10 includes only numerous medical imaging data of a specific type. Accordingly, the learning efficiency and learning quality when learning is performed using such a learning data set improve.

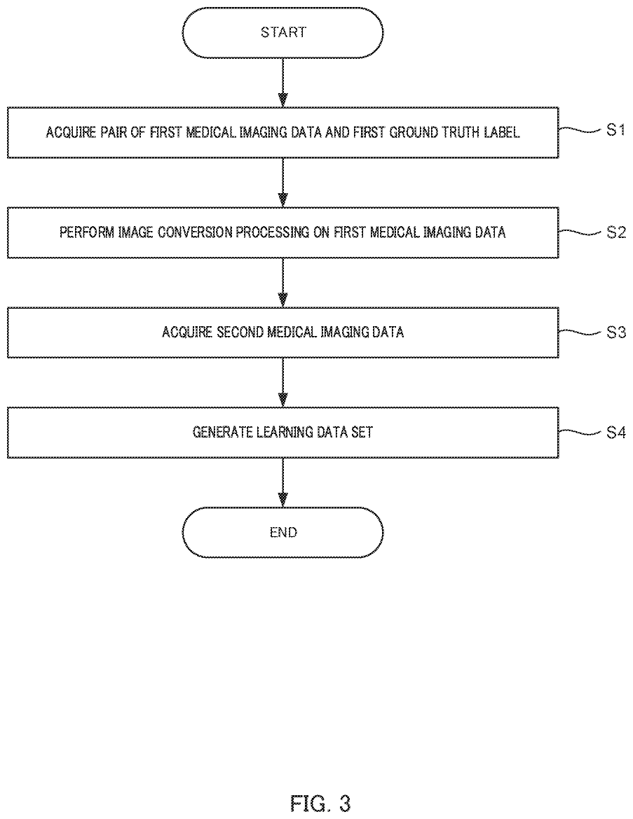

FIG. 3 is a flowchart provided for describing an operation example of learning data set generation device 10.

In step S1, learning data set generation device 10 acquires a data set, which is a pair of first medical imaging data and a first ground truth label, from outside.

In step S2, learning data set generation device 10 performs predetermined image conversion processing on the first medical imaging data to generate second medical imaging data.

In step S3, learning data set generation device 10 acquires the second medical imaging data generated in step S2.

In step S4, learning data set generation device 10 generates the pair of the second medical imaging data and the first ground truth label as a learning data set.

Although the operation of learning data set generation device 10 stops after the processing in step S4 in the flowchart illustrated in FIG. 3, the operation in FIG. 3 may be repeated, for example, in a predetermined cycle.

Learning data set generation device 10 may be implemented by a computer such as a server device, a personal computer (PC), a smartphone, or a tablet terminal. In addition, various functions of learning data set generation device 10 are implemented by the computer executing a program.

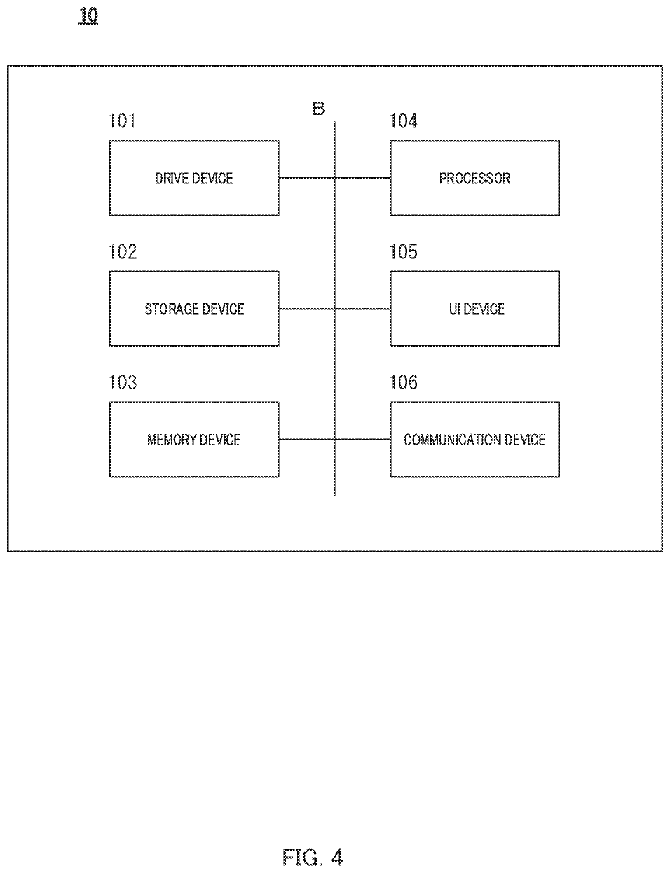

FIG. 4 is a diagram illustrating an example of a hardware configuration of learning data set generation device 10. Learning data set generation device 10 includes drive device 101, storage device 102, memory device 103, processor 104, user interface (UI) device 105, and communication device 106, which are connected to each other via bus B.

The programs or instructions that implement the various functions and processing to be described later in learning data set generation device 10 may be stored in a removable storage medium such as a compact disk-read only memory (CD-ROM) or a flash memory. When the storage medium is set in drive device 101, a program or instruction is installed in storage device 102 or memory device 103 from the storage medium via drive device 101. Note that, the program or instruction is not necessarily required to be installed from the storage medium, but may be downloaded from an external device via a network or the like.

Storage device 102 is implemented by a hard disk drive or the like and stores, together with an installed program or instruction, a file, data, or the like used for the execution of the program or instruction.

Memory device 103 is implemented by a random access memory, a static memory, or the like, and, when a program or instruction is activated, reads a program, an instruction, data, or the like from storage device 102 and stores the read program, instruction, data, or the like. Storage device 102, memory device 103, and the removable storage medium may be collectively referred to as a non-transitory tangible storage medium.

Processor 104 may be implemented by one or more central processing units (CPUs), graphics processing units (GPUs), processing circuitry, or the like, which may be formed of one or more processor cores, and executes various functions and processes of learning data set generation device 10, which will be described later, according to programs, instructions, data such as parameters necessary for executing the programs or instructions, and the like, stored in memory device 103.

User interface (UI) device 105 may be formed of an input device(s) such as a keyboard, a mouse, a camera, and a microphone, an output device(s) such as a display, a speaker, a headset, and a printer, and an input/output device(s) such as a touch screen, and implements an interface between the user and learning data set generation device 10. For example, the user operates learning data set generation device 10 by operating a graphical user interface (GUI) displayed on a display or a touch screen with a keyboard, a mouse, or the like.

Communication device 106 is implemented by various communication circuits that execute wired and/or wireless communication processing with an external device or a communication network such as the Internet, a local area network (LAN), or a cellular network.

The above-described hardware configuration is, however, merely an example, and learning data set generation device 10 according to the present disclosure may be implemented by any other appropriate hardware configuration.

### <Machine Learning Device 20>

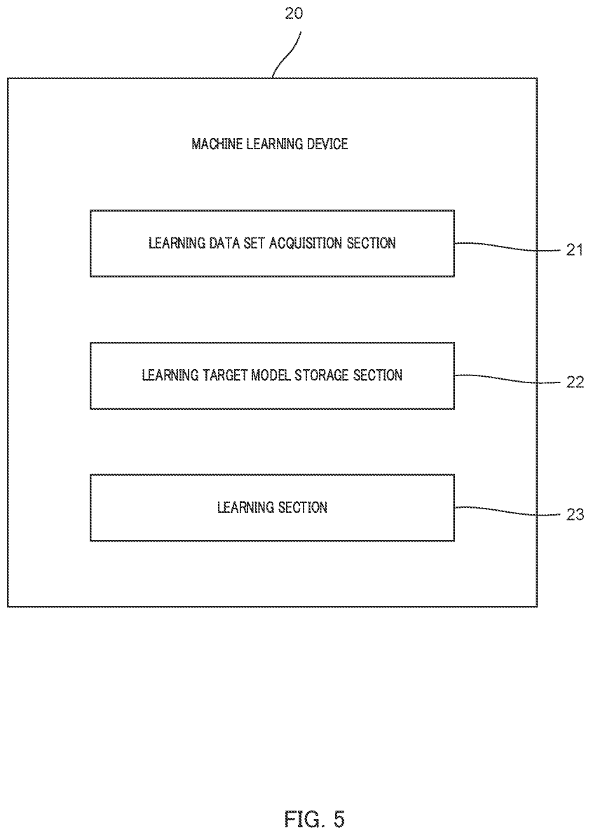

Next, machine learning device 20 will be described. FIG. 5 is a block diagram exemplifying a configuration of machine learning device 20. As illustrated in FIG. 5, machine learning device 20 includes learning data set acquisition section 21, learning target model storage section 22, and learning section 23.

Learning data set acquisition section 21 acquires a learning data set outputted from learning data set generation device 10.

Learning target model storage unit 22 stores a machine learning model (image diagnosis model) which is a target for learning using a learning data set.

Learning section 23 trains the image diagnosis model stored in learning target model storage section 22 by using the learning data set. Thus, the image diagnosis model is subjected to, for example, learning for outputting an image diagnosis result as an inference result in response to an input of medical imaging data.

Learning section 23 outputs the learned image diagnosis model to image diagnosis device 30.

### [Image Diagnosis Device 30]

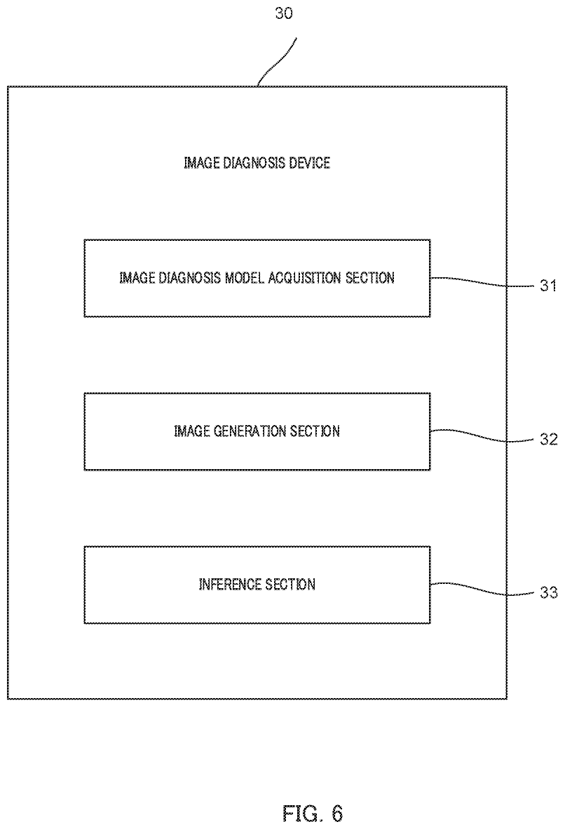

Next, image diagnosis device 30 will be described. Image diagnosis device 30 is an example of an image processing device of the present disclosure. FIG. 6 is a block diagram exemplifying a configuration of image diagnosis device 30. As illustrated in FIG. 6, image diagnosis device 30 includes image diagnosis model acquisition section 31, image generation section 32, and inference section 33.

Image diagnosis model acquisition section 31 acquires the learned image diagnosis model outputted from machine learning device 20.

Image generation section 32 generates new third medical imaging data. The third medical imaging data generated by image generation section 32 is image data of the same type as the second medical imaging data included in the learning data set used for learning the image diagnosis model. In other words, learning data set generation device 10 determines the type of the second medical imaging data, into which the first medical imaging data is converted, according to the type of the third medical imaging data generated by image generation section 32 of image diagnosis device 30.

In a case where image generation section 32 generates, for example, ultrasound image data as the third medical imaging data, the second medical imaging data included in the learning data set generated by learning data set generation device 10 is also ultrasound image data. In this case, the image diagnosis model on which machine learning device 20 performs machine learning is a machine learning model that outputs an image diagnosis result as an inference result in response to an input of ultrasound image data.

Inference section 33 performs image diagnosis on the third medical imaging data generated by image generation section 32 using the learned image diagnosis model acquired by image diagnosis model acquisition section 31. Specifically, inference section 33 inputs the third medical imaging data into the image diagnosis model, acquires an image diagnosis result as an inference result, and outputs the image diagnosis result. The image diagnosis result includes, for example, the position of a region of interest and the position and type of an affected area in the ultrasound image data.

Note that, although image diagnosis device 30 has been described as a modality capable of generating the third medical imaging data in the present embodiment, the present disclosure is not limited thereto. For example, image diagnosis device 30 may not generate the third medical imaging data and in this case acquires the third medical imaging data from outside and outputs an image diagnosis result by using an image diagnosis model. Even in this case, image diagnosis device 30 can assist the medical worker in image diagnosis, which is suitable.

### Operation and Effects

As described above, according to image diagnosis system 100 according to the embodiment of the present disclosure, learning data set generation device 10 is capable of generating second medical imaging data, which appears to be medical imaging data of the same type as third medical imaging data generated by image diagnosis device 30, from first medical imaging data which is medical imaging data of another type, and using the second medical imaging data for learning of machine learning device 20. For this reason, even in a case where the third medical imaging data is medical imaging data of a type for which it is difficult to prepare a large amount of learning data, a large amount of second medical imaging data can be obtained with little labor by converting first medical imaging data of various other types into the second medical imaging data.

Further, learning data set generation device 10 generates a learning data set by applying the first ground truth label corresponding to the first medical imaging data, which is the conversion source, to the second medical imaging data obtained by the image conversion processing. That is, since a new learning data set can be generated by using an already existing ground truth label as it is, the learning data set can be efficiently obtained. As described above, since a large number of learning data sets can be prepared with relatively little labor, it is possible to generate image diagnosis models with high diagnosis performance by performing machine learning using the learning data sets in machine learning device 20. Further, this improves the accuracy of image diagnosis performed by image diagnosis device 30 that performs image diagnosis by using the learned image diagnosis model.

In particular, in a case where the third medical imaging data is medical imaging data generated by using a specific ultrasound probe, for example, medical imaging data generated by using an ultrasound probe which is not a major ultrasound probe in terms of manual technique is very small as data, and the size of the database is small. It is significantly difficult to cause an image diagnosis model for performing image diagnosis based on medical imaging data generated by using such an ultrasound probe to be efficiently learned. According to image diagnosis system 100 according to the embodiment of the present disclosure, in a case where machine learning is performed on an image diagnosis model for performing image diagnosis based on medical imaging data generated by using such a non-major ultrasound probe, a large number of learning data sets can be easily generated based on medical imaging data generated by using a major ultrasound probe, and thus, the efficiency of machine learning can be significantly improved and an image diagnosis model capable of performing highly accurate image diagnosis can be efficiently learned.

Alternatively, in a case where the third medical imaging data is ultrasound image data, it may be difficult to draw a subject, or it may be difficult to perform labeling (such as designating whether an affected area is included in an image, or surrounding a particular region in an image) for reasons such as a poor SN ratio or difficulty in applying a probe. In such a case, for example, by using, as the first medical imaging data, MRI image data generated by using a nuclear magnetic resonance imaging device that is likely to have a higher SN ratio, it is possible to reduce the labor for assigning a first ground truth label to the first medical imaging data. Further, it is possible to generate a high-quality learning data set by performing the image conversion processing on first medical imaging data to generate second medical imaging data which appears to be ultrasound image data, and setting a pair of the second medical imaging data and a first ground truth label as a learning data set.

One embodiment of the present disclosure described above is a learning data set generation device that generates a learning data set, a machine learning device that performs machine learning on an image diagnosis model by using the learning data set, an image diagnosis device that outputs an image diagnosis result by using the learned image diagnosis model, and an image diagnosis system that includes these components. The image diagnosis system of the present disclosure may be, for example, an ultrasound image diagnosis system capable of handling ultrasound image data.

Hereinabove, the examples of the present disclosure have been described in detail, but the present disclosure is not limited to the above-described specific embodiments, and various modifications and changes can be made within the scope of the gist of the present disclosure described in the claims.

Although embodiments of the present invention have been described and illustrated in detail, the disclosed embodiments are made for purposes of illustration and example only and not limitation. The scope of the present invention should be interpreted by terms of the appended claims.

What is claimed is:

1. A learning data set generation method causing a computer to execute:
acquiring second medical imaging data generated by image conversion processing on first medical imaging data; and
generating a pair of the second medical imaging data and a first ground truth label as a learning data set, the first ground truth label being a ground truth label for the first medical imaging data.

2. The learning data set generation method according to claim 1, wherein
the image conversion processing is executed by an image conversion model, the image conversion model being a model on which machine learning has been performed.

3. The learning data set generation method according to claim 1, wherein
the image conversion processing is processing in which medical imaging data corresponding to a second medical imaging device is generated as the second medical imaging data by converting the first medical imaging data generated by a first medical imaging device, the second medical imaging device being a medical imaging device different in type from the first medical imaging device.

4. The learning data set generation method according to claim 3, wherein
the first medical imaging device and the second medical imaging device include, respectively, ultrasound probes of types different from each other.

5. The learning data set generation method according to claim 3, wherein:
the first medical imaging device is a nuclear magnetic resonance imaging device, and
the second medical imaging device is an ultrasound imaging device.

6. The learning data set generation method according to claim 1, wherein
the image conversion processing is executed by an image conversion model using a convolutional neural network or an attention mechanism.

7. The learning data set generation method according to claim 1, wherein
the image conversion processing is image conversion processing in which a contour position of the first medical imaging data and a contour position of the second medical imaging data do not change.

8. The learning data set generation method according to claim 1, wherein
the learning data set includes first learning data and second learning data, the first learning data being formed of the first medical imaging data and the first ground truth label, the second learning data being formed of a pair of the second medical imaging data and the first ground truth label.

9. A machine learning model causing a computer to execute:
acquiring medical imaging data; and
outputting an inference result with respect to the medical imaging data, wherein
machine learning using a learning data set formed of a pair of second medical imaging data and a first ground truth label has been performed on the machine learning model, the second medical imaging data generated by image conversion processing on first medical imaging data, the first ground truth label being a ground truth label for the first medical imaging data.

10. An image processing device, comprising an inference section that outputs a first inference result obtained by inputting third medical imaging data into the machine learning model according to claim 9.

11. The image processing device according to claim 10, further comprising an image generation section that generates the third medical imaging data.

12. The image processing device according to claim 11, wherein
the image generation section generates ultrasound image data, the ultrasound image data generated based on a reflected ultrasound wave with respect to an ultrasound wave transmitted to a subject.

13. A learning data set generation device, comprising:
an image data acquisition section that acquires second medical imaging data generated by image conversion processing on first medical imaging data; and
a generation section that generates a pair of the second medical imaging data and a first ground truth label as a learning data set, the first ground truth label being a ground truth label for the first medical imaging data.

14. A machine learning device, comprising:
a learning data set acquisition section that acquires a learning data set formed of a pair of second medical imaging data and a first ground truth label, the second medical imaging data generated by image conversion processing on first medical imaging data, the first ground truth label being a ground truth label for the first medical imaging data; and
a learning section that performs machine learning on a machine learning model by using the learning data set.

15. An image diagnosis system, comprising:
a learning data set generation device that generates a learning data set formed of a pair of second medical imaging data and a first ground truth label, the second medical imaging data generated by image conversion processing on first medical imaging data, the first ground truth label being a ground truth label for the first medical imaging data;
a machine learning device that performs machine learning on a machine learning model by using the learning data set; and
an image diagnosis device that outputs an inference result obtained by inputting third medical imaging data into the machine learning model on which the machine learning has been performed.

16. A non-transitory computer-readable recording medium storing therein a program to be executed by a computer, the program causing a computer to implement:
an acquisition function of acquiring second medical imaging data generated by image conversion processing on first medical imaging data; and
a generation function of generating, as a learning data set, a pair of the second medical imaging data and a first ground truth label, the first ground truth label being a ground truth label for the first medical imaging data.
